(19) United States
(12) Patent Application Publication
(10) Pub. No.: US 2015/0050319 A1
(43) Pub. Date: Feb. 19, 2015

(54) NATURAL SAPONIN-BASED SYNTHETIC IMMUNOADJUVANTS

(71) Applicant: THE UAB RESEARCH FOUNDATION, Birmingham, AL (US)

(72) Inventor: Pengfei Wang, Hoover, AL (US)

(73) Assignee: THE UAB RESEARCH FOUNDATION, Birmingham, AL (US)

(21) Appl. No.: 14/386,988

(22) PCT Filed: Mar. 12, 2013
(86) PCT No.: PCT/US13/30415

§ 371 Date: Sep. 22, 2014

Related U.S. Application Data
(60) Provisional application No. 61/614,744, filed on Mar. 23, 2012.

(51) Int. Cl. A61K 39/39; C07H 15/256

(52) U.S. Cl. CPC A61K 39/39; C07H 15/256; A61K 2039/55583
USPC 424/278.1; 536/18.1; 514/33

(57) ABSTRACT

The present disclosure encompasses QS-21-based structurally-defined adjuvants to address the need for stronger, safer, and easier-to-access adjuvants. The new compositions can provide tools for addressing long-standing mechanistic questions concerning saponin immune-potentiation through structure-activity-relationship (SAR) studies. Most advantageously, the compounds of the disclosure may be formulated into pharmaceutically acceptable compositions, including vaccines that may be delivered to a subject human or animal subject. The compounds can then act as, for example, an adjuvant to augment an immunological response to a vaccine immunogen.

### CROSS-REFERENCE TO RELATED APPLICATIONS

This application claims priority to U.S. Provisional Patent Application Ser. No. 61/614,744 entitled “NATURAL SAPONIN-BASED SYNTHETIC IMMUNOADJUVANTS” and filed Mar. 23 2012, the entirety of which is hereby incorporated by reference.

### STATEMENT REGARDING FEDERALLY SPONSORED RESEARCH OR DEVELOPMENT

This invention was made with government support under NIH Grant No. R03AI099407 awarded by the U.S. National Institutes of Health of the United States government. The government has certain rights in the invention.

### TECHNICAL FIELD

The present disclosure is generally related to novel synthetic saponin-based immunoadjuvants.

### BACKGROUND

Vaccination is one of the most successful medical practices since its invention 200 years ago, and has been successful in eradicating many severe infectious diseases (Plotkin S A (2005) Nature Med. Supplement, 11, S5-S11; Mortellaro & Ricciardi-Castagnoli (2011) Immun. Cell. Bio. 89, 332-339). However, the current state of vaccine development is not adequate to meet some emerging, re-emerging or persistent challenges. Infectious diseases are still responsible for one-fifth of all deaths worldwide, killing at least 11 million people every year (Kieny M P, Girard M P (2005) Vaccine, 23, 5705-5707). Conventional vaccinology has failed in fighting many pathogens due to either high antigen variability or poor understanding of protection mechanisms. Effective prophylactic vaccines are still needed for prevention of persisting infectious diseases such as human immune-deficiency virus (HIV), tropical parasitic diseases (malaria, leishmaniases and schistosomiasis), tuberculosis (TB), and influenza. Effective therapeutic vaccines are also needed for both chronic infectious diseases and non-infectious chronic disorders (Sela & Hilleman (2004) Proc. Natl. Acad. Sci. USA, 101, Suppl 2, 14559). Moreover, new pathogens and drug resistant strains (wild type or bioengineered for bioterrorism) continue to emerge. Recent threats of SARS, Hepatitis C, avian influenza, yellow fever, dengue, Japanese encephalitis, and West Nile fever challenge vaccine development (Hombach et al., (2005) Vaccine, 23, 2689-2695). For many infectious diseases expected to emerge, no cure is currently available (Rappuoli R. (2004) Nat. Med. 10, 1171-1185; Jones et al., (2008) Nature, 451, 990). Rapid development of new vaccines against persistent or new diseases is an urgent priority. While the conventional empirical approach has been necessary and may have paved the way to future success, it is vital to have new approaches that make full use of ever increasing immunological knowledge. To this end, the recent development of vaccines to combat cancer and vaccine-resistant infectious diseases has relied heavily on structurally defined subunit antigen constructs. Although there are many advantages of these vaccine candidates, the refined antigens are often less immunogenic, which necessitates the use of adjuvants. Adjuvants are substances applied to enhance the ability of a vaccine to elicit strong and durable immune responses to a co-administered antigen (Kensil et al., (2006) Vaccine Adjuvants, Ed. Hackett, C J, Harn D A, 221-234; Leroux-Roels G (2010) Vaccine, 28S, C25-C36; Brunner et al., (2010) Immun. Lett. 128, 29-35).

Adjuvants play crucial roles in vaccine development. For instance, (i) adjuvants can be used to enhance the immune response, allowing for the use of otherwise impotent antigens or allowing for effective vaccination for poor responders (e.g., older or immune-compromised patients); (ii) adjuvants may decrease the amount of antigen required to invoke a protective response, thus potentially reducing side effects resulting from high doses and reducing costs of the vaccine employing rare, difficult-to-produce, or expensive antigens; (iii) adjuvants improve the ability for rapid response to vaccine crisis with low antigen supply (e.g., during an epidemic or preparing for bioterrorism); and (iv) adjuvants have a profound effect on the nature of the immune response, and can bias the immune system toward either a Th1 or a Th2 type response. This is extremely important for the development of subunit vaccines against cancers and intracellular pathogens (e.g. HIV, TB and malaria) when a sufficiently potent cytotoxic T lymphocyte (CTL) response to purified antigens without toxicity issues is desired. Although using adjuvants has been introduced into vaccinology for many decades, only a handful of adjuvants have both sufficient potency and acceptable toxicity for clinical investigation. The alum-based adjuvants (aluminum hydroxide or phosphate) remain the most commonly used adjuvants in human vaccines (Pascual et al., (2006) Vaccine, 24, S88-89). A major limitation of an alum-based adjuvant is that it stimulates only Th2 immunity, which is effective for neutralizing vaccines but ineffective for vaccines requiring a Th1 or mixed Th1/Th2 responses. Adjuvant discovery and evaluation will continue to play a significant role in developing vaccines of the future.

QS-21 is a unique adjuvant, not only stimulating Th2 immunity but also Th1 immune response with the production of antigen-specific CTL (Kensil et al., (2004) Frontiers in Bioscience, 9, 2972-2988; Kensil et al., (2006) Vaccine Adjuvants, Ed. Hackett, C J, Harn D A, 221-234). It is one of the most promising new adjuvants and has been evaluated in more than 80 recent and ongoing vaccine clinical trials. QS-21 is a mixture of two isomers (QS-21ₐₚᵢ and QS-21_{xyl} in a ratio of 2:1) obtained from the tree bark of Quillaja saponaria (QS) Molina (Kensil et al., (1991) J. Immun. 146, 431-437). These two isomers differ in the terminal sugar unit of the linear tetrasaccharide segment connected to the central triterpene (i.e. the quillaic acid (QA) core) at the C-28 carboxyl group, as shown in FIG. 1 of the disclosure). However, they have the same adjuvanticity and toxicity. Although QS-21 remains the immunostimulant of choice in many cancer and infectious disease vaccine trials, the inherent drawbacks (e.g. its scarcity, difficulty and low-yield in purification, chemical instability, and dose-limiting toxicity) prevent it from wide use, especially in the production of reliable human vaccines other than for life-threatening diseases, such as HIV infection or cancer. There is a real and on-going need for stable and accessible adjuvants with better chemical, pharmacological, and immunological properties than those of naturally occurring QS-21.

### SUMMARY

The present disclosure provides embodiments of compounds that may be advantageous as adjuvants. The disclosure further encompasses methods of their synthesis that are fewer in steps than current methods for synthesizing QS-21-derived compounds. Accordingly, one aspect of the disclosure, therefore, provides embodiments of a QS-21 derivative having the formula (I):

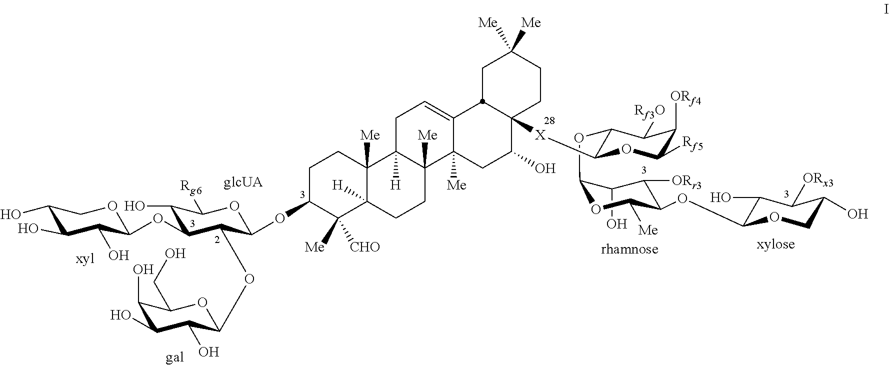

wherein X can be —C(O)O—, —C(O)NH—, —(CH₂)ₐO—, —CO₂(CH₂)ₐO—, or —C(O)NH(CH₂)ₐO—, wherein a can be an integer from 1 to 5; R_{g6} can be H, Me, alkyl, —COR₁, or —CH₂OR′; Rₓ₃, Rᵣ₃, and R_{f3} are each independently H or a monosaccharide; R_{f4} can be H, acetyl, or a monosaccharide; R_{f5} can be H, Me, alkyl, monosaccharide, —COR₁, or —CH₂OR′; and where R₁ can be OH, OR′, —NR₂(CH₂)ₘR₃, —NR₂[(CH₂)ₙ—O—(CH₂)ₚ]ₓR₃, —NR₂[(CH₂)ₙ—NH—(CH₂)ₚ]ₓR₃, —NR₂[(CH₂)ₙ—S—(CH₂)ₚ]ₓR₃, —NR₂[(CH₂)ₙ—C(O)—NH—(CH₂)ₚ]ₓR₃, or —NR₂[(CH₂)ₙ—NH—C(O)—(CH₂)ₚ]ₓR₃; R′ can be H, —(CH₂)ₘR₃, —[(CH₂)ₙ—O—(CH₂)ₚ]ₓR₃, —[(CH₂)ₙ—NH—(CH₂)ₚ]ₓR₃, —[(CH₂)ₙ—S—(CH₂)ₚ]ₓR₃, —[(CH₂)ₙ—C(O)—NH—(CH₂)ₚ]ₓR₃, or —[(CH₂)ₙ—NH—C(O)—(CH₂)ₚ]ₓR₃, R₂ can be H, alkyl, —(CH₂)ₘR₃, —[(CH₂)ₙ—O—(CH₂)ₚ]ₓR₃, —[(CH₂)ₙ—NH—(CH₂)ₚ]ₓR₃, —[(CH₂)ₙ—S—(CH₂)ₚ]ₓR₃, —[(CH₂)ₙ—C(O)—NH—(CH₂)ₚ]ₓR₃, or —[(CH₂)ₙ—NH—C(O)—(CH₂)ₚ]ₓR₃, R₃ can be CH₃, Ph, COOH, CHO, CONH₂, OH, SH, NH₂, a monosaccharide, a disaccharide, a trisaccharide, or a tetrasaccharide; and m can be an integer from 1 to 14, n and p are each independently integers from 0 to 12, and x can be an integer from 1 to 12;

In embodiments of this aspect of the disclosure, Rₓ₃ can be H, apiose or xylose.

In embodiments of this aspect of the disclosure, R_{f3} can be H, or glucose.

In embodiments of this aspect of the disclosure, the QS-21 derivative can have the formula (II):

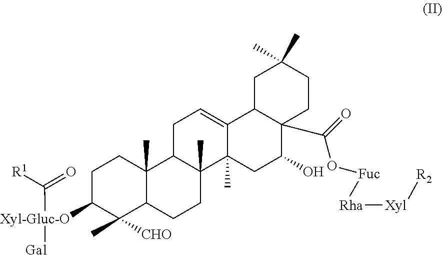

where R₁ can be OBn, —NH(CH₂)ₘCH₃, —NH(CH₂)ₙ—O—(CH₂)ₚ, CO₂H—(CH₂)_{q}—NHCO—(CH₂)ᵣ, CO₂H—(CH₂)ₛ, trisaccharide-(CH₂)ₜ, or CHO—(CH₂)ᵤ, wherein m=can be an integer from 1 to 14; n, p, q, r, s, t and u are each independently integers from 1 to 12, where, when R₁ is OBn, the xylose and galactose are optionally blocked with triethylsilyl (TES), and where the trisaccharide can be fucose-rhamnose-xylose-R₂, where R₂ can be H, xylose or apiose, and where the fucose-rhamnose-xylose-R₂ can be optionally blocked with acetyl groups (Ac).

In embodiments of this aspect of the disclosure, the R_{g6} is —COR₁, with R₁ selected from:

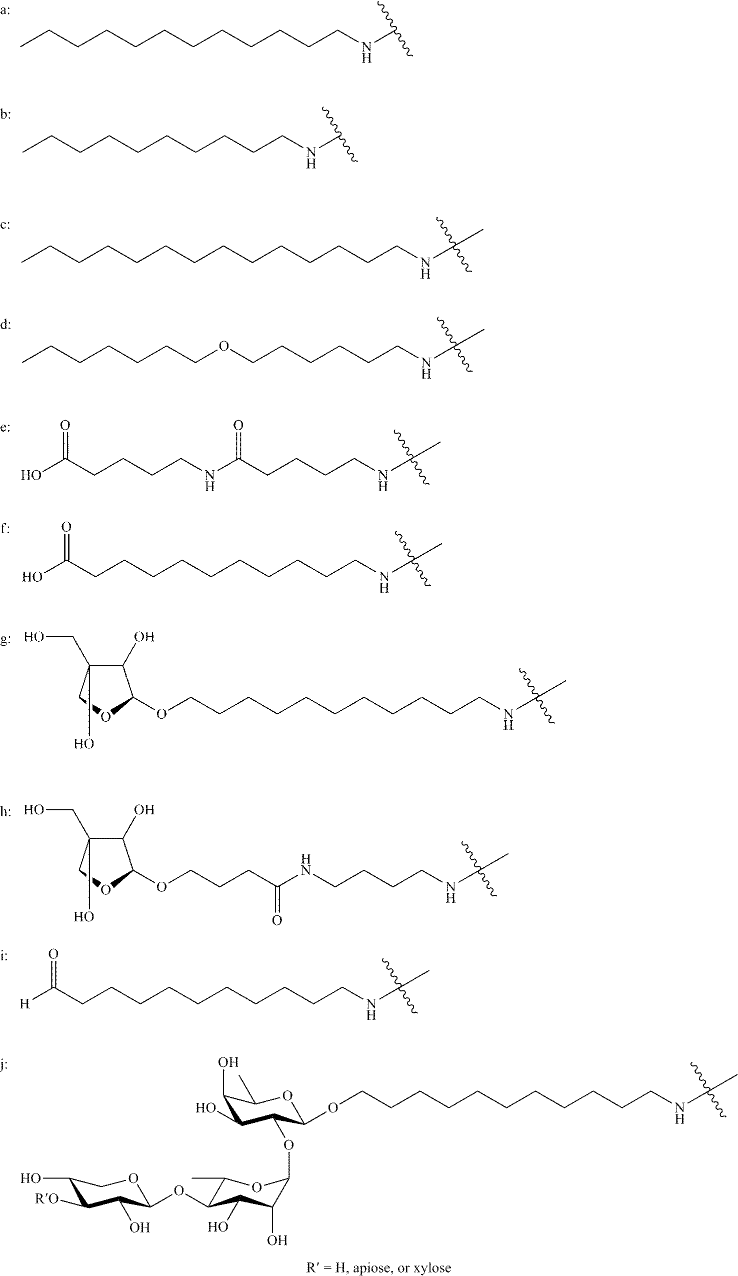

Another aspect of the disclosure encompasses embodiments of a QS-21 derivative having the formula (III):

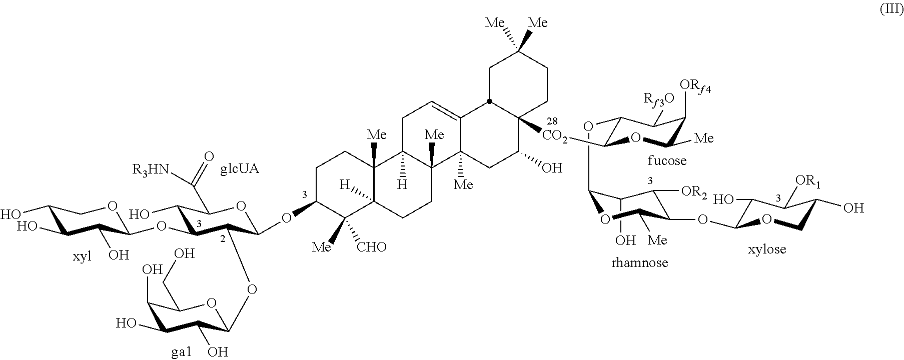

wherein

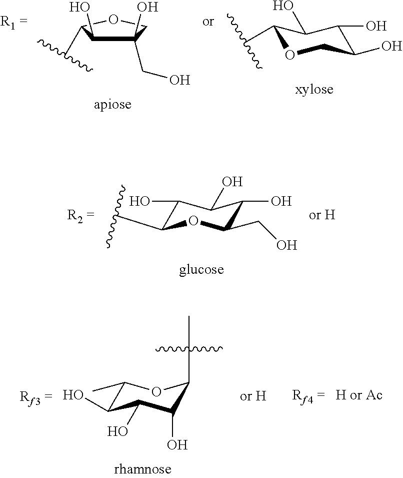

### and R₃ is any of:

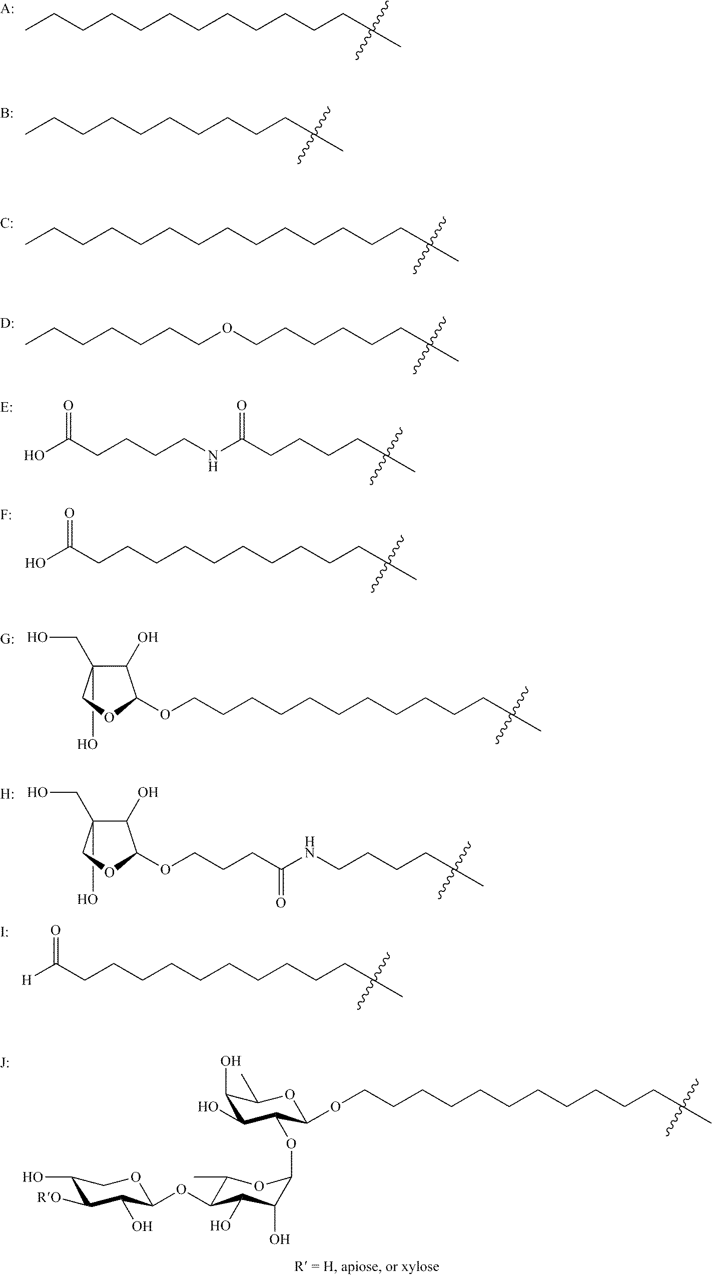

Still another aspect of the disclosure encompasses embodiments of a pharmaceutically acceptable composition comprising at least one of the compounds according to the disclosure.

In embodiments of this aspect of the disclosure, the pharmaceutically composition can be formulated as a vaccine.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1 illustrates the structure of the naturally occurring immune adjuvant QS-21 and the chemically reactive sites causing chemical instability of the natural product.

FIG. 2 illustrates the base formula (I) for QS-21-based synthetic adjuvants of the disclosure.

FIG. 3 illustrate an example structure of synthetic adjuvant candidate (1).

FIGS. 4A and 4B illustrate embodiments of side-chains of the adjuvants of the disclosure.

FIG. 5 illustrates the scheme for the synthesis of the tetrasaccharide components of the compounds according to the disclosure

FIG. 6 illustrates the scheme for the synthesis of alternative tetrasaccharide components of the compounds according to the disclosure.

FIGS. 7A and 7B illustrate a scheme for the synthesis of compound 1.

The drawings are described in greater detail in the description and examples below.

The details of some exemplary embodiments of the methods and systems of the present disclosure are set forth in the description below. Other features, objects, and advantages of the disclosure will be apparent to one of skill in the art upon examination of the following description, drawings, examples and claims. It is intended that all such additional systems, methods, features, and advantages be included within this description, be within the scope of the present disclosure, and be protected by the accompanying claims.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. Patent law and can mean “includes,” “including,” and the like; “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Prior to describing the various embodiments, the following definitions are provided and should be used unless otherwise indicated.

### DEFINITIONS

In describing and claiming the disclosed subject matter, the following terminology will be used in accordance with the definitions set forth below.

The term “alkyl” as used herein refers to saturated monovalent hydrocarbon groups having straight, branched, or cyclic moieties (including fused and bridged bicyclic and spirocyclic moieties), or a combination of the foregoing moieties. For an alkyl group to have cyclic moieties, the group must have at least three carbon atoms.

The term “protecting group” refers to any chemical moiety that may be attached to a compound, including an intermediary compound in a reaction, thereby preventing undesirable modification of the structure to which the protecting group is attached. Their introduction and removal are described, for example, in “Protective Groups in Organic Synthesis”, T. W. Greene et al., John Wiley & Sons Inc., Second Edition 1991. Suitable protecting group donor compounds, e.g. amino group protecting agents, are well-known to a skilled person and may include, but are not limited to, anhydrides, halides, carbamates or N-hydroxysuccinimides, carboxybenyl, and methoxy (MeO). It will be recognized that it may be preferred or necessary to prepare such a compound in which a functional group is protected using a conventional protecting group, then to remove the protecting group, to provide a compound of the present disclosure. The details concerning the use of protecting groups in accordance with the present invention are known to those skilled in the art.

The term “saccharide” as used herein refers to monosaccharides that are the most basic units of biologically important carbohydrates. They are the simplest form of sugar and are usually colorless, water-soluble, crystalline solids. Examples of monosaccharides include, but are not limited to, glucose (dextrose), fructose (levulose), galactose, xylose, rhamnose, apiose, ribose, and the like. Monosaccharides of use in the synthesis of the compounds of the disclosure are well know in the art and are contemplated to be incorporated into the embodiments of the disclosure. Monosaccharides are the building blocks of disaccharides such as sucrose. Further, each carbon atom that supports a hydroxyl group (except for the first and last) is chiral, giving rise to a number of isomeric forms all with the same chemical formula. For instance, galactose and glucose are both aldohexoses, but have different chemical and physical properties. With few exceptions (e.g., deoxyribose), monosaccharides have this chemical formula: Cx(H₂O)y, where conventionally x≧3.

The term “pharmaceutically acceptable” as used herein refers to a compound or combination of compounds that while biologically active will not damage the physiology of the recipient human or animal to the extent that the viability of the recipient is comprised. Preferably, the administered compound or combination of compounds will elicit, at most, a temporary detrimental effect on the health of the recipient human or animal is reduced.

The term “pharmaceutically acceptable carrier” as used herein refers to a diluent, adjuvant, excipient, or vehicle with which a probe of the disclosure is administered and which is approved by a regulatory agency of the Federal or a state government or listed in the U.S. Pharmacopeia or other generally recognized pharmacopeia for use in animals, and more particularly in humans. Such pharmaceutical carriers can be liquids, such as water and oils, including those of petroleum, animal, vegetable or synthetic origin, such as peanut oil, soybean oil, mineral oil, sesame oil and the like. The pharmaceutical carriers can be saline, gum acacia, gelatin, starch paste, talc, keratin, colloidal silica, urea, and the like. When administered to a patient, the composition of the disclosure and pharmaceutically acceptable carriers can be sterile. Water is a useful carrier when the probe is administered intravenously. Saline solutions and aqueous dextrose and glycerol solutions can also be employed as liquid carriers, particularly for injectable solutions. Suitable pharmaceutical carriers also include excipients such as glucose, lactose, sucrose, glycerol monostearate, sodium chloride, glycerol, propylene, glycol, water, ethanol and the like. The present compositions, if desired, can also contain minor amounts of wetting or emulsifying agents, or pH buffering agents. The present compositions advantageously may take the form of solutions, emulsion, sustained-release formulations, or any other form suitable for use.

### Description

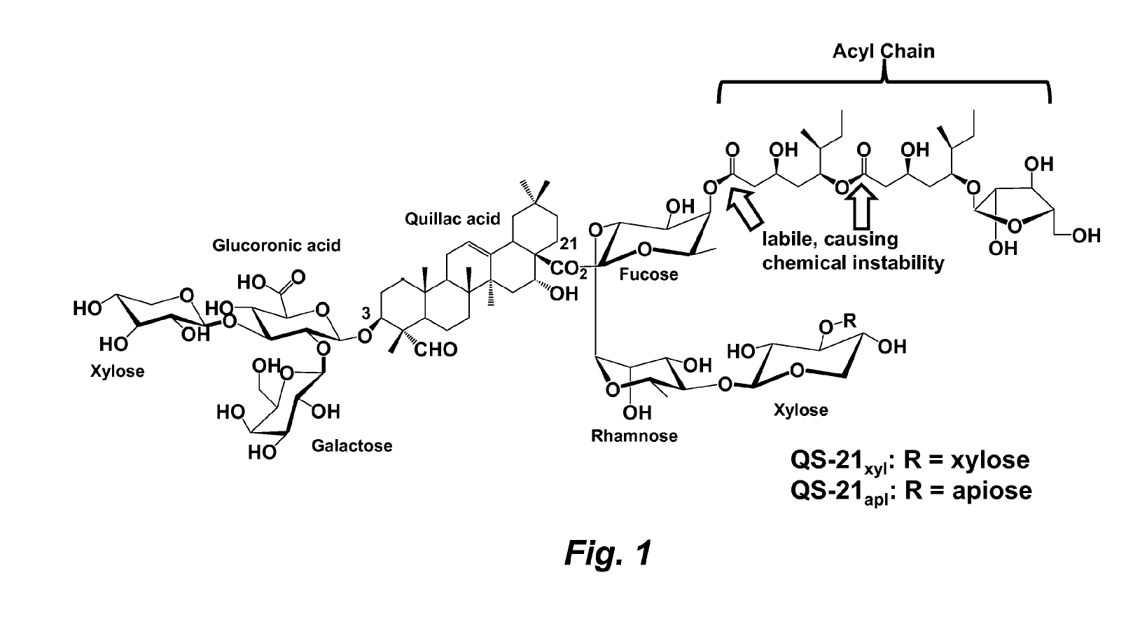

Structural modification of QS-21 for improved adjuvant activity and toxicity profiles is an attractive method to develop a better and structurally defined new generation of tailored adjuvants. Recently, derivatization of QS-21 has been carried out. The strategy was to make hydrolytically stable analogs to mimic QS-21 by replacing the unstable ester linkages of the acyl side chain (as indicated in FIG. 1) with more stable amide linkages. The new synthetic analogs exhibited similar or better in vivo immunostimulating potencies compared with naturally occurring QS-21.

The present disclosure encompasses QS-21-based structurally-defined adjuvants to address the need for stronger, safer, and easier-to-access adjuvants. The compositions of the disclosure have broader impacts in the field beyond clinical applications of the products. The new compositions of the disclosure can provide tools for addressing long-standing mechanistic questions concerning saponin immune-potentiation through structure-activity-relationship (SAR) studies. Most advantageously, the compounds of the disclosure may be formulated into pharmaceutically acceptable compositions, including vaccines that may be delivered to a subject human or animal subject. The compounds can then act as, for example, an adjuvant to augment an immunological response to a vaccine immunogen.

Despite QS-21 being among the world's leading adjuvant candidates and its decades-long use in numerous clinical trials, the mechanism of action of QS-21 is not fully understood. Existing data have excluded QS-21's depot effect (Kensil C R (1996) Crit. Rev. Ther. Drug Carrier Syst. 13, 1-55) and its effect as an agonist of Toll-like receptors 2 and 4 (TLR-2 and TLR-4) to activate innate immune responses by recognizing pathogen-associated molecular patterns (Pink &, Kieny (2004) Vaccine, 22, 2097-102). While these findings are significant, many mechanistic questions remain to be answered. For example, QS-21 improves B cell response but it is unknown if this is through a direct effect or via APC/T cell stimulation (Fogg et at, 2007). QS-21 improves antigen presentation by APC and therefore optimize the T cell response but it is not known if it is correlated to its ability to intercalate into cell membranes, leading to the formation of pores (O'Hagan et al., (2001) Biomol. Engineer. 18: 69-85). The poor availability and chemical stability of QS-21 complicates the mechanistic studies. The limitations of QS-21 highlight the needs for similar adjuvants with improved properties, which are addressed by the embodiments of the present disclosure.

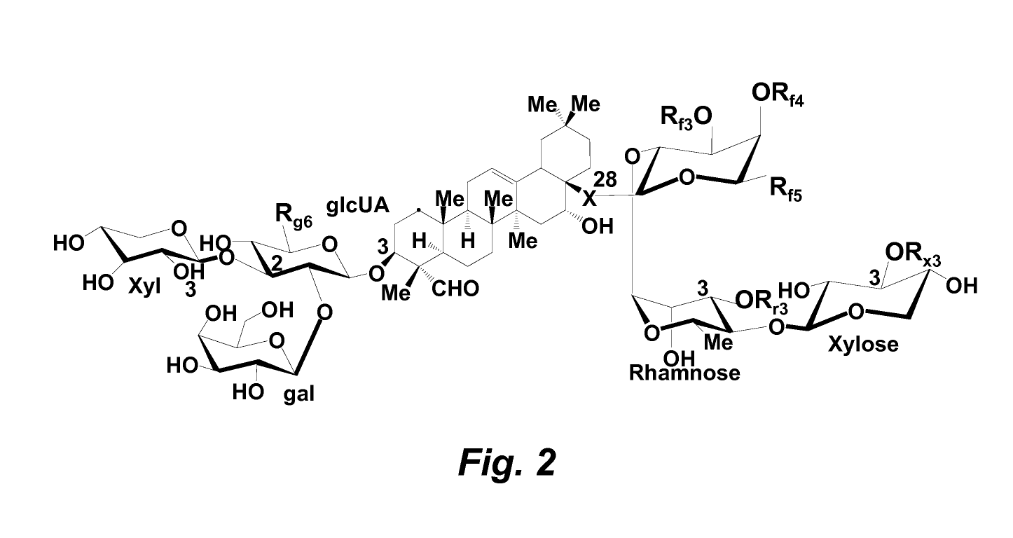

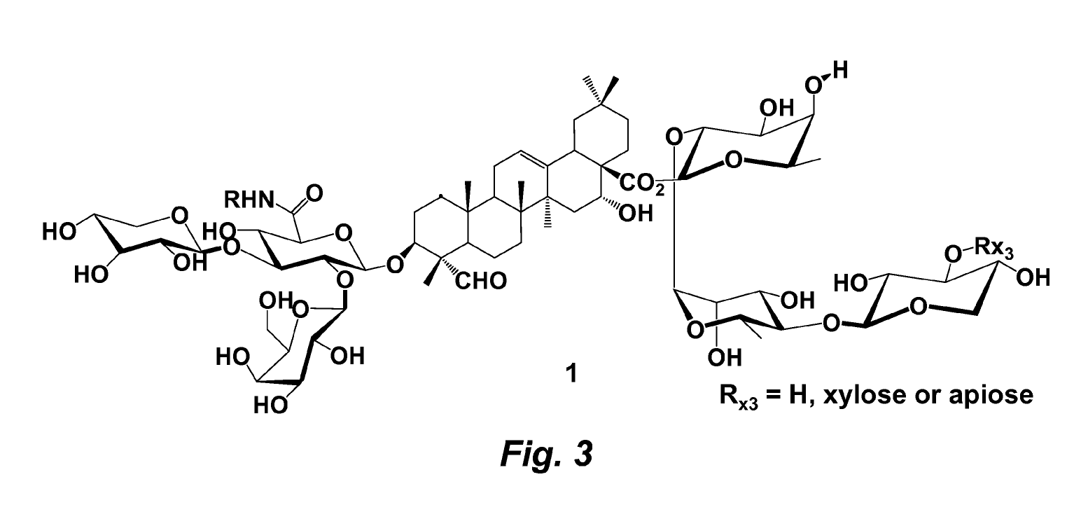

Accordingly, the novel QS-21-based compounds of the disclosure that are contemplated as synthetic adjuvants, such as derivatives of the general Formula I, FIG. 2, and of formulas II and III (below) address both stability and toxicity issues that limit the use of natural QS-21 in vaccine development. The strategy differs from earlier synthetic efforts of making hydrolytically-stable natural product mimics by keeping the side chain at its original position (i.e. the C3 of the fucosyl unit of the linear tetrasaccharide segment). In the advantageous embodiments of the disclosure, there is no side chain at this position. Instead, a new chain is added on the other side of the molecule (i.e. at the glucuronic acid site). The new side chain can be a variety of structures suitable for SAR exploration. The design, therefore, can useful as adjuvants of significantly lowered toxicity while maintaining potent adjuvant activity.

The disclosure provides a new synthetic approach for step-economical carbohydrate synthesis for the expeditious access to carbohydrate segments of various adjuvant candidates. This new methodology improves the overall efficiency for the syntheses of complex saponin structures and, therefore, advantageously, new adjuvants can be prepared at a low cost.

The lipophilic acyl chain of QS-21, as shown in FIG. 1 likely is responsible for the toxicity and instability under physiological conditions since chain removal results in a significant decrease in toxicity with a concomitant loss in the ability to stimulate a lymphoproliferative response and CTL production (Marciani et al., (2000) Vaccine, 18, 3141-3151; Liu et al., (2011) Vaccine, 29, 2037-2043). The instability of the acyl chain is disadvantageous for vaccine formulation since storage would lead to a loss of the vaccines' capacity to stimulate a Th1 immune response and their capacity to produce antigen-specific CTL, which are required in the case for anti-viral, anti-parasitic or anti-cancer vaccines.

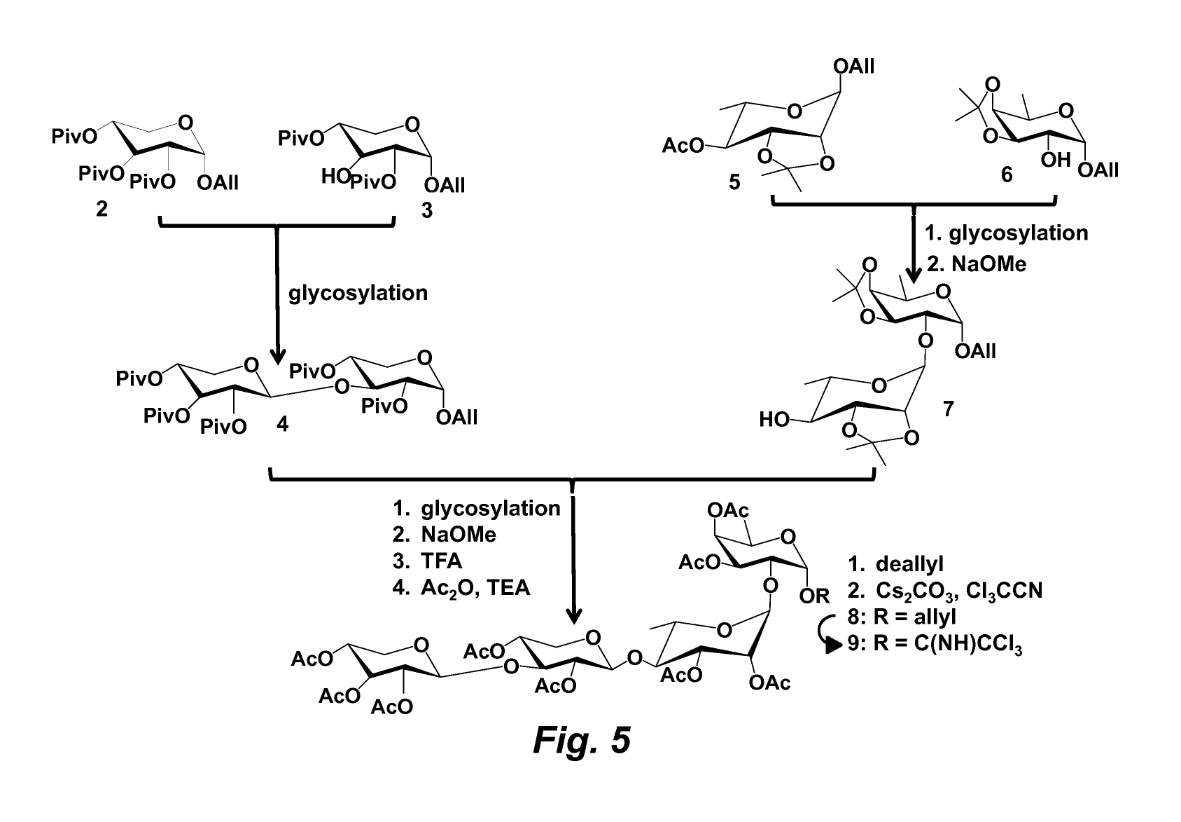

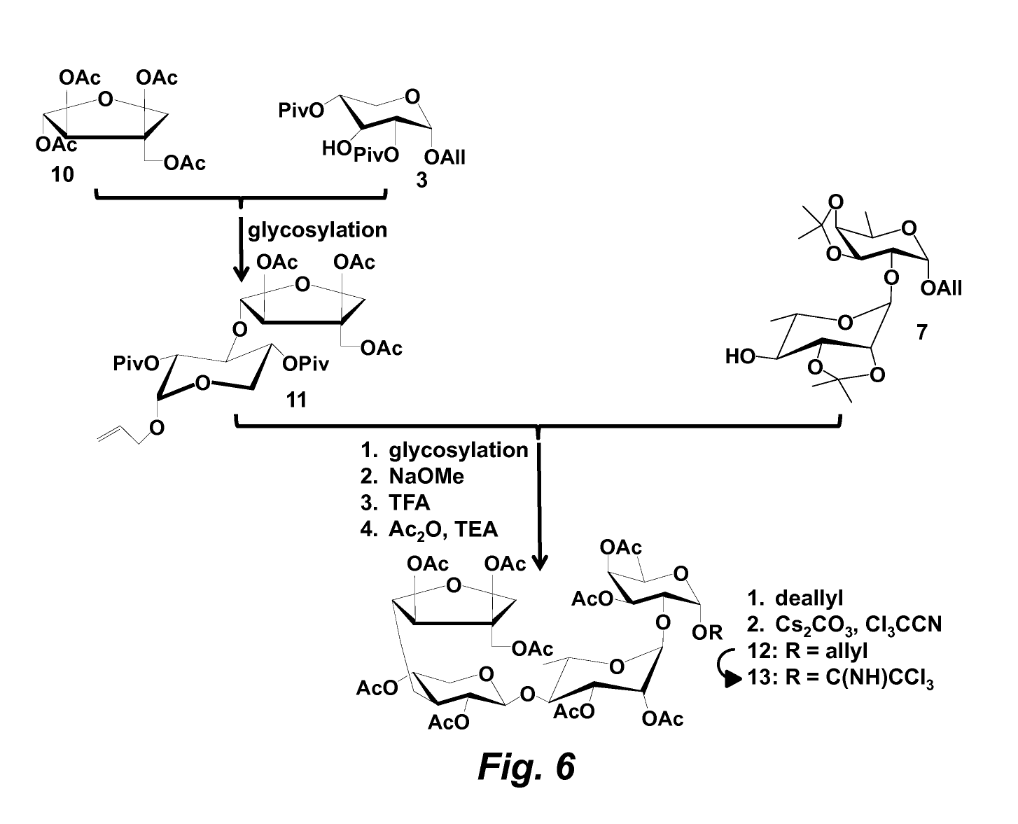

A simple and mild glycosylation reaction employing only allyl glycosides as building blocks has been developed. The efficacy and efficiency of this new approach in synthesizing important carbohydrate antigens has been demonstrated. This approach is equally efficient in synthesizing the different linear tetrasaccharides of QS-21 Aₐₚᵢ and QS-21A_{xyl}. These tetrasaccharides are indispensable components of the proposed synthetic adjuvants. Methods used to synthesize the compositions of the present disclosure are depicted schematically in FIGS. 5-7.

Accordingly, the necessary building blocks are available (i.e. 8, 9, 12, 14, 15, and 16) for synthesizing the compounds of the disclosure. This is a substantially shorter saponin adjuvant synthesis compared with the original QS-21ₐₚᵢ synthesis (more than 70 steps) of QS-21 and analog synthesis (more than 40 steps).

For the synthesis, the feasibility of the synthetic route to the most challenging tetrasaccharide targets (i.e. 8, 9, 12, 14) has been verified. With the building blocks available, the final product can be synthesized in a few steps. Thus, the two major components, the linear tetrasaccharide (either 9 or 13) and the quillaic acid-trisaccharide conjugate (14) can be connected to produce 15, a key intermediate for the divergent synthesis of a series of other adjuvants. Coupling of dodecylamine with 15 will produce 16. Globe deprotection by using known procedures (Adams et al., 2010, incorporated herein by reference in its entirety) will generate the desired compound 1A (i.e., 1 with Rx3=xyloside and R=dodecyl).

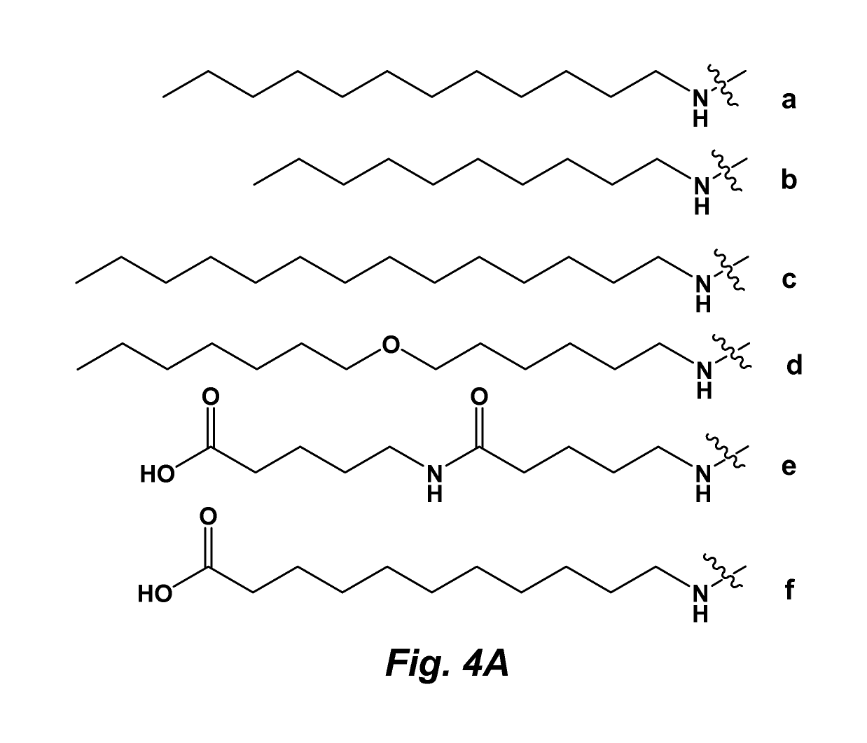

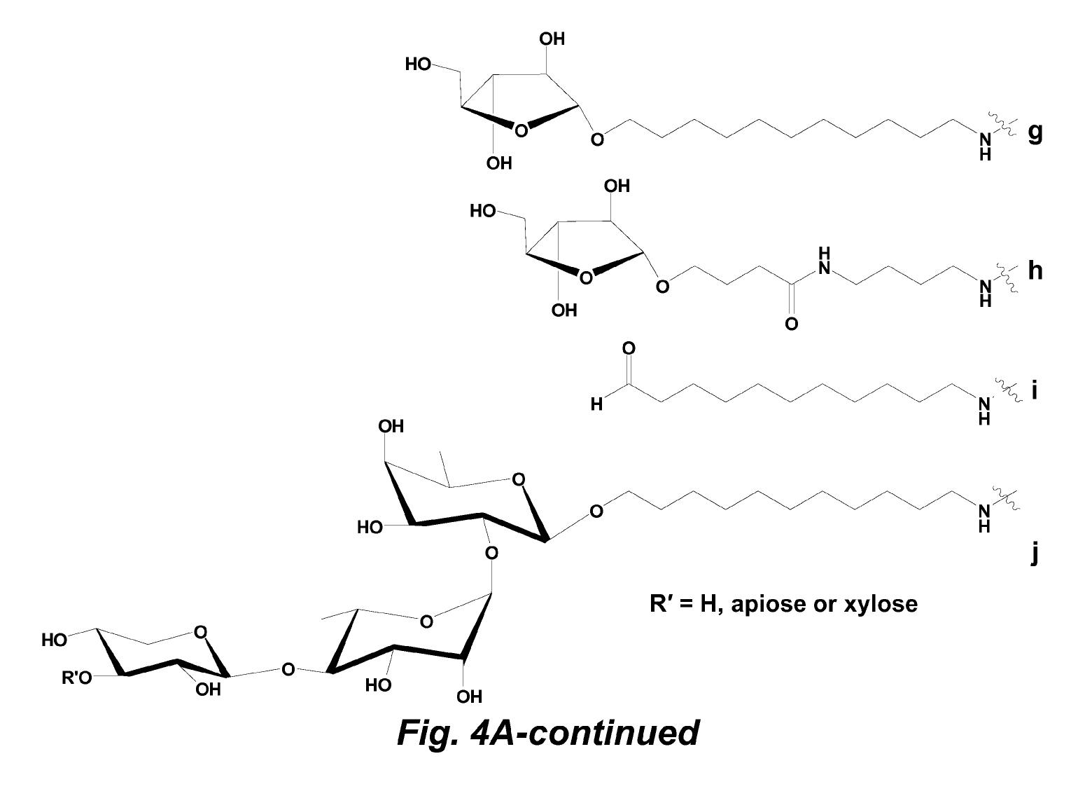

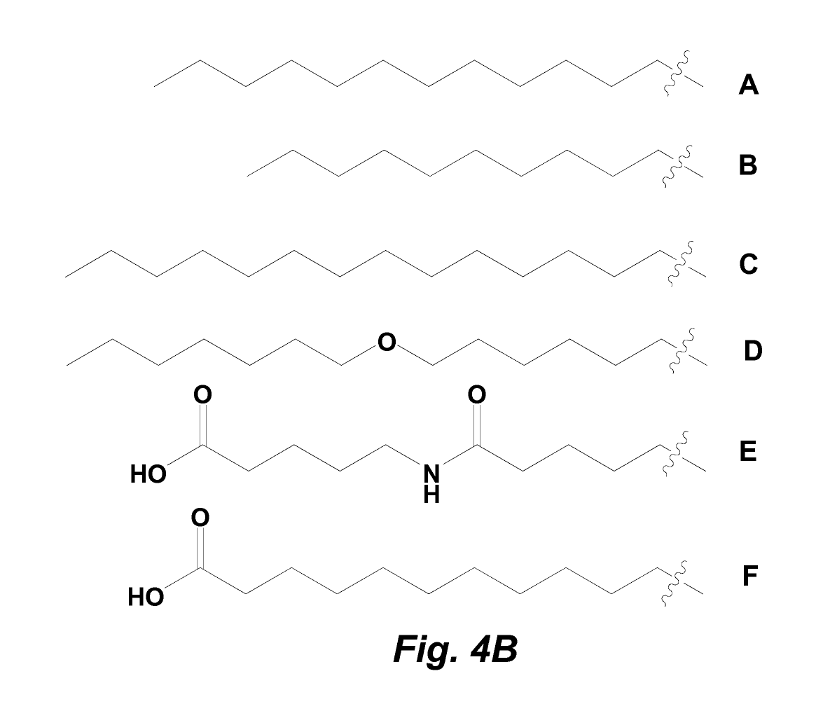

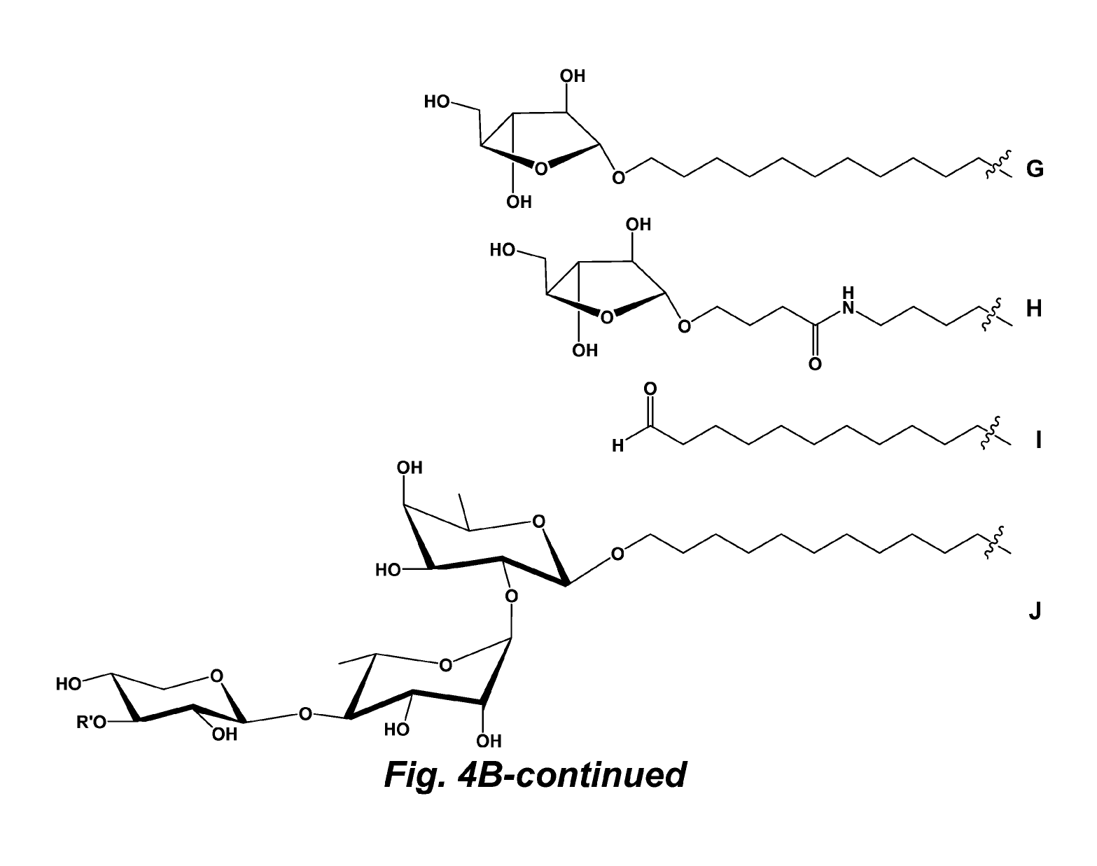

Since known SAR results suggested that derivatization of the carboxylic group of the glucuronic acid unit of QS-21 is feasible in maintaining its adjuvanticity, different chains other than the dodecyl amide group of 1A can be prepared. The common intermediate 15 accumulates in the synthesis of 1A. Divergent synthesis from 15 by using the similar synthetic approach will lead to new compounds that can be usefully screened on the basis of current SAR knowledge of QS-21 for use as adjuvants. Exemplary embodiments of side-chains for use in the compounds of the disclosure are shown, for example, but not intended to be limiting, in FIG. 4.

It is known that the side chain of QS-21 is essential to CTL production, albeit the mechanism remains unknown. The lower adjuvanticity of the GPI-0100 species was attributed to its increased hydrophobic properties relative to those of natural saponins, leading to micelle formation (Marciani et al., 2000). Preventing micelle formation was anticipated to increase the adjuvant's availability to stimulate antigen-presenting cells (APC). For a systematic evaluation of the chain's length effect on adjuvanticity, two adjuvants, 1B (2 carbons shorter) and 1C (2 carbons longer than that of 1A) can be useful in this regard. The compound 1D would have a lower tendency of micelle formation than 1C owing to its ether side chain.

In 1G and 1H, a terminal arabinose unit is used to mimic the natural side chain. Some other, but not intended to be limiting structures are shown in FIG. 4A.

One aspect of the disclosure, therefore, provides embodiments of a QS-21 derivative having the formula (I):

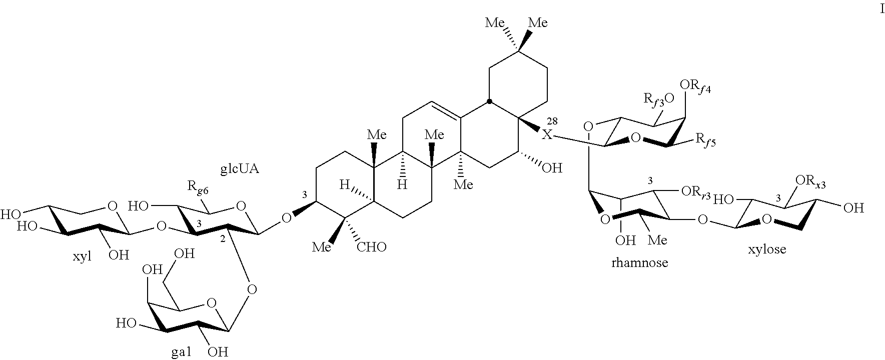

wherein X can be —C(O)O—, —C(O)NH—, —(CH₂)ₐO—, —CO₂(CH₂)ₐO—, or —C(O)NH(CH₂)ₐO—, wherein a can be an integer from 1 to 5; R_{g6} can be H, Me, alkyl, —COR₁, or —CH₂OR′; Rₓ₃, Rᵣ₃, and R_{f3} are each independently H or a monosaccharide; R_{f4} can be H, acetyl, or a monosaccharide; R_{f5} can be H, Me, alkyl, monosaccharide, —COR₁, or —CH₂OR′; and where R₁ can be OH, OR′, —NR₂(CH₂)ₘR₃, —NR₂[(CH₂)ₙ—O—(CH₂)ₚ]ₓR₃, —NR₂[(CH₂)ₙ—NH—(CH₂)ₚ]ₓR₃, —NR₂[(CH₂)ₙ—S—(CH₂)ₚ]ₓR₃, —NR₂[(CH₂)ₙ—C(O)—NH—(CH₂)ₚ]ₓR₃, or —NR₂[(CH₂)ₙ—NH—C(O)—(CH₂)ₚ]ₓR₃; R′ can be H, —(CH₂)ₘR₃, —[(CH₂)ₙ—O—(CH₂)ₚ]ₓR₃, —[(CH₂)ₙ—NH—(CH₂)ₚ]ₓR₃, —[(CH₂)ₙ—S—(CH₂)ₚ]ₓR₃, —[(CH₂)ₙ—C(O)—NH—(CH₂)ₚ]ₓR₃, or —[(CH₂)ₙ—NH—C(O)—(CH₂)ₚ]ₓR₃, R₂ can be H, alkyl, —(CH₂)ₘR₃, —[(CH₂)ₙ—O—(CH₂)ₚ]ₓR₃, —[(CH₂)ₙ—NH—(CH₂)ₚ]ₓR₃, —[(CH₂)ₙ—S—(CH₂)ₚ]ₓR₃, —[(CH₂)ₙ—C(O)—NH—(CH₂)ₚ]ₓR₃, or —[(CH₂)ₙ—NH—C(O)—(CH₂)ₚ]ₓR₃, R₃ can be CH₃, Ph, COOH, CHO, CONH₂, OH, SH, NH₂, a monosaccharide, a disaccharide, a trisaccharide, or a tetrasaccharide; and m can be an integer from 1 to 14, n and p are each independently integers from 0 to 12, and x can be an integer from 1 to 12;

In embodiments of this aspect of the disclosure, Rₓ₃ can be H, apiose or xylose.

In embodiments of this aspect of the disclosure, R_{f3} can be H, or glucose.

In embodiments of this aspect of the disclosure, the QS-21 derivative can have the formula (II):

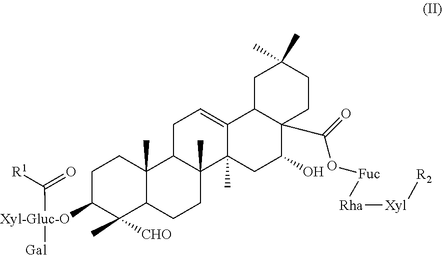

where R₁ can be OBn, —NH(CH₂)ₘCH₃, —NH(CH₂)ₙ—O—(CH₂)ₚ, CO₂H—(CH₂)_{q}—NHCO—(CH₂)ᵣ—, CO₂H—(CH₂)ₛ, trisaccharide-(CH₂)ₜ, or CHO—(CH₂)ᵤ, wherein m=can be an integer from 1 to 14; n, p, q, r, s, t and u are each independently integers from 1 to 12, where, when R₁ is OBn, the xylose and galactose are optionally blocked with triethylsilyl (TES), and where the trisaccharide can be fucose-rhamnose-xylose-R₂, where R₂ can be H, xylose or apiose, and where the fucose-rhamnose-xylose-R₂ can be optionally blocked with acetyl groups (Ac).

In embodiments of this aspect of the disclosure, the R_{g6} is —COR₁, with R₁ selected from:

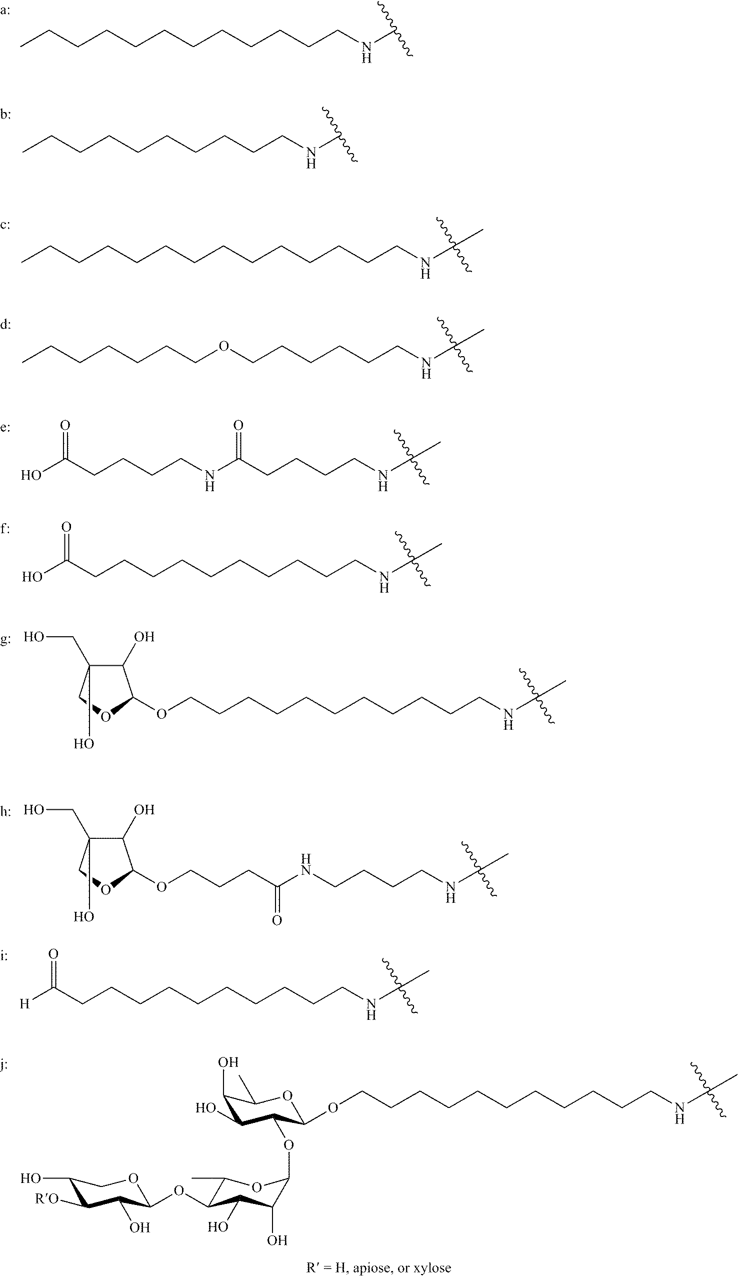

Another aspect of the disclosure encompasses embodiments of a QS-21 derivative having the formula (III):

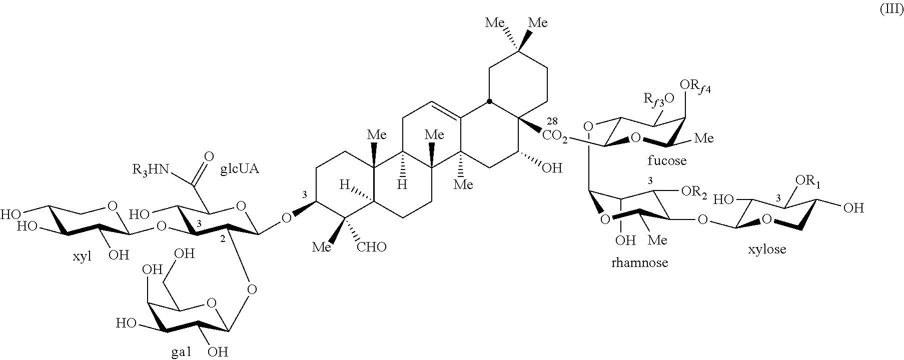

wherein

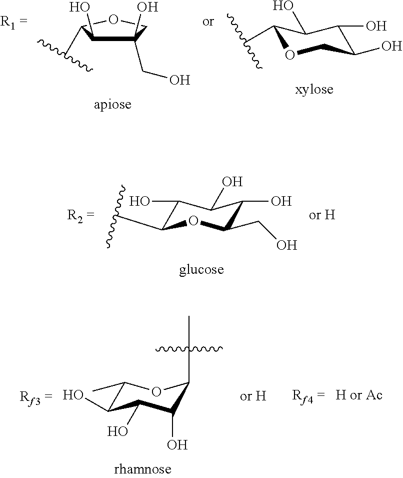

### and R₃ is any of:

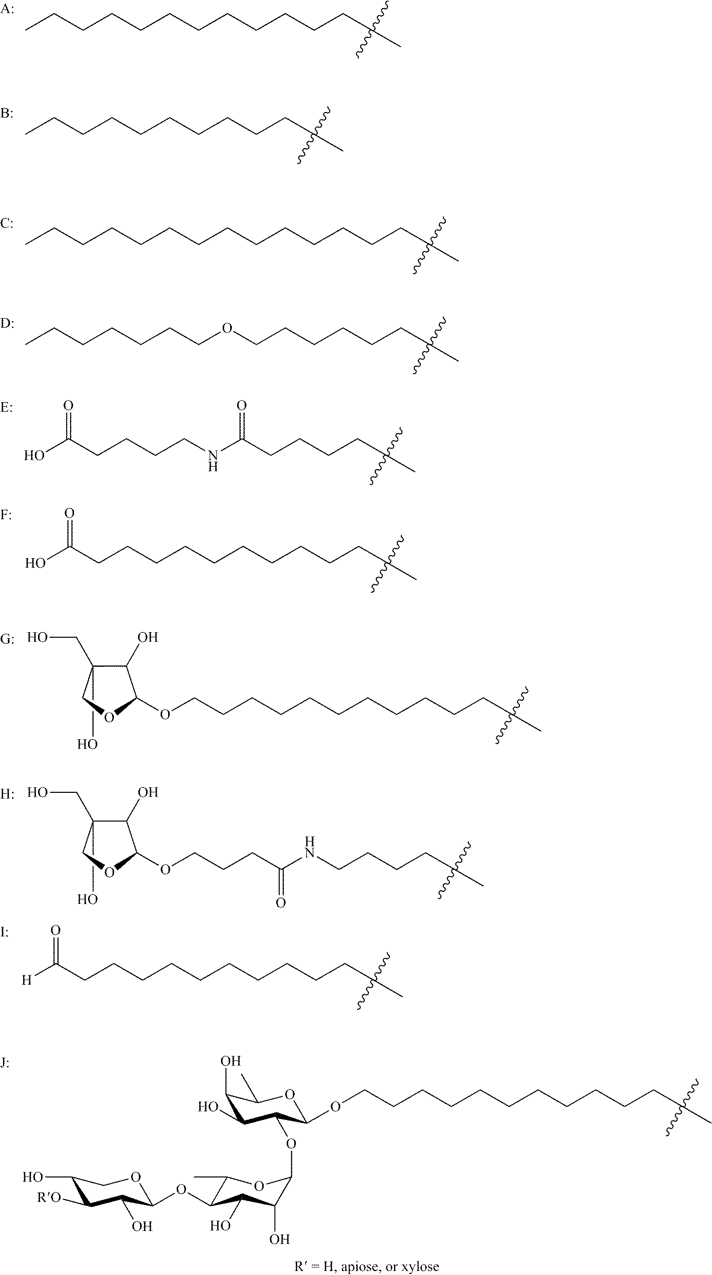

Still another aspect of the disclosure encompasses embodiments of a pharmaceutically acceptable composition comprising at least one of the compounds according to the disclosure.

In embodiments of this aspect of the disclosure, the pharmaceutically composition can be formulated as a vaccine.

The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All publications recited herein are hereby incorporated by reference in their entirety.

It should be emphasized that the embodiments of the present disclosure, particularly, any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure, and the present disclosure and protected by the following claims.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, ±2%, ±3%, ±4%, ±5%, ±6%, ±7%, ±8%, ±9%, or ±10%, or more of the numerical value(s) being modified.

### EXAMPLES

### Example 1

Synthesis:

Both xyloside building blocks 2 and 3 were isolated from the reaction of allyl xyloside with 2.2 equiv. of pivaloyl chloride. Glycosylation with the xyloside donor 2 and acceptor 3 led to the disaccharide 4 in 71% yield. The reaction started with isomerization of the anomeric allyl group of 2 by using hydrogen-activated [Ir(COD)(PMePh₂)₂]PF₆, followed by subsequent treatment of the obtained prop-1-enyl donor with NIS/TfOH in the presence of the acceptor 3. The same glycosylation procedure was applied to the rhamnoside donor 5 and the fucoside acceptor 6. After removal of the acetyl group at 4-O of the rhamnoside unit, the disaccharide 7 was obtained in 82% yield. Glycosylation reaction with the donor 4 and the acceptor 7 resulted in the tetrasaccharide 8 after replacing all the protecting groups with the acetyl protection. The allyl tetrasaccharide 8 was then converted to the imidate donor 9. Another tetrasaccharide donor 13 was prepared in a similar manner as depicted in Scheme 2.

As shown in Scheme 3, with the linear tetrasaccharide (9) and the quillaic acid-trisaccharide conjugate (14), the compound 15 with the desired anomeric stereochemistry was prepared in 72% yield. Debenzylation under hydrogenolysis conditions followed by coupling of the released carboxyl group and dodecylamine led to the fully protected product 16 in 97% yield. The final global deprotection consisted of two steps, i.e., removal of all triethylsiyl groups with TFA/H₂O (4:1 v:v) and removal of acetyl groups with K₂CO₃ in methanol. The final product 1A was obtained in 85% yield.

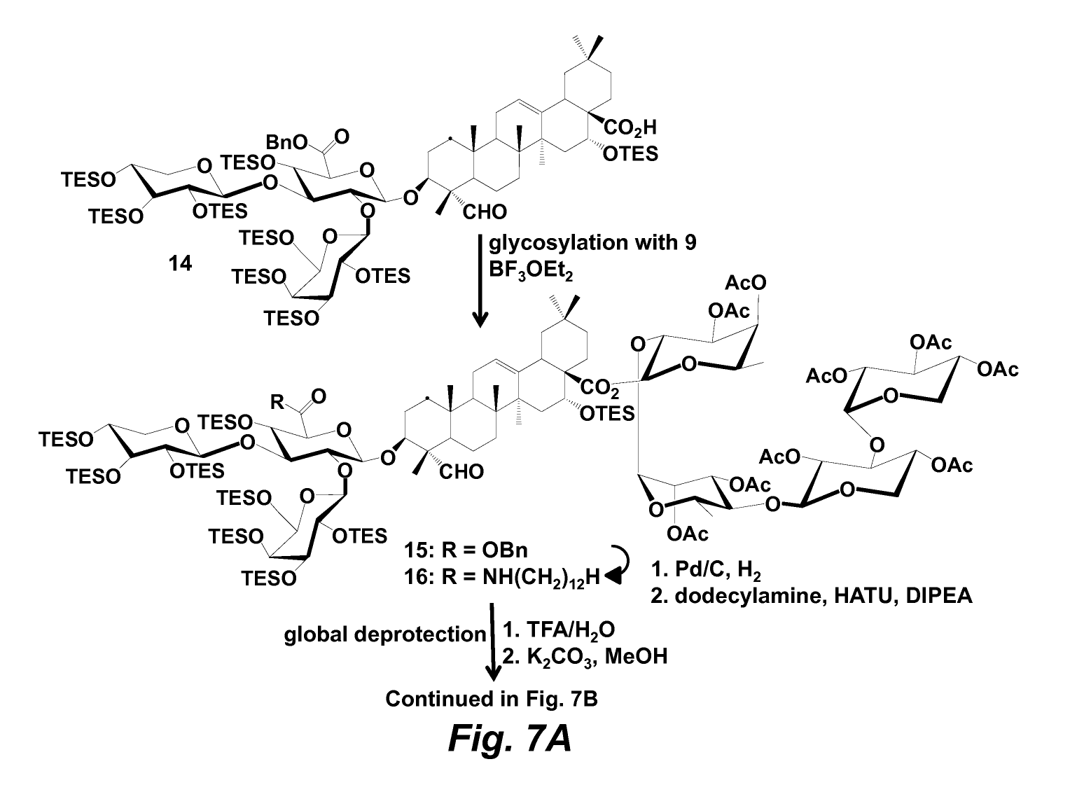

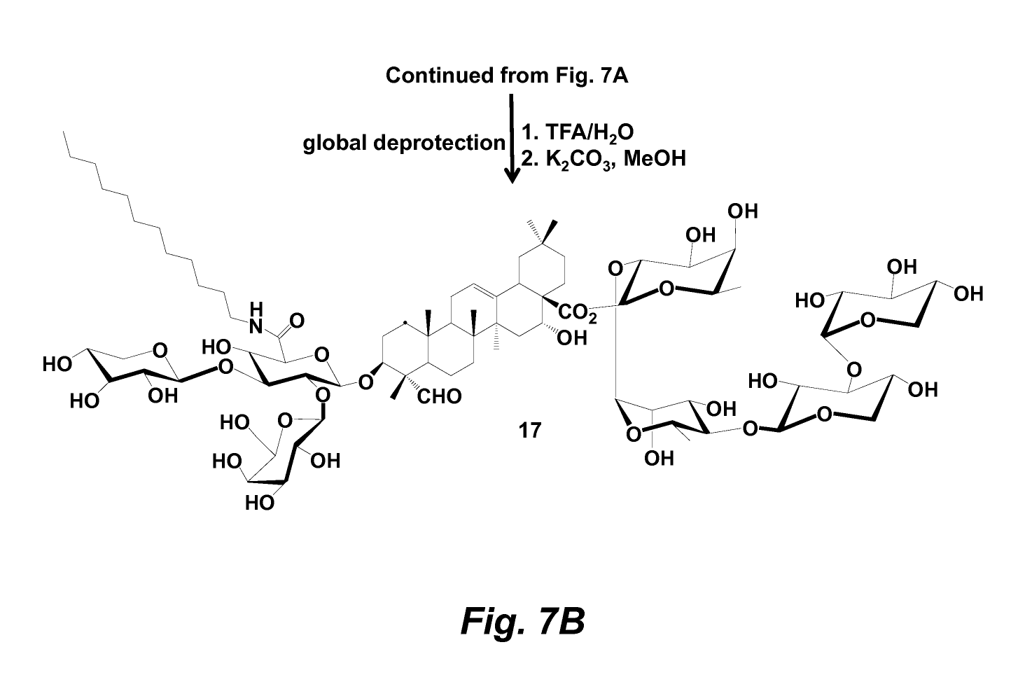

The building block 14, a quillaic acid-trisaccharide conjugate (FIG. 7) was prepared in three steps from commercially available saponins by using standard procedures (Higuchi et al., (1987) Phytochemistry, 26, 229-235; Deng et al., (2008) J. Am. Chem. Soc. 130, 5860-5861, incorporated herein by reference in their entireties). Semi-purified QS-bark extracts have a complex mixture of over 100 distinct saponins. Many of these saponins share the same quillaic acid-trisaccharide core structure. It is, therefore, possible to obtain the quillaic acid-trisaccharide conjugate in high yields from QS-bark extracts and convert it to the desired building block 14 (about 0.25 g of 13 from 1 g of extracts) (Deng et al., (2008) J. Am. Chem. Soc. 130, 5860-5861).

### Example 2

A QS-21 derivative having the formula (I):

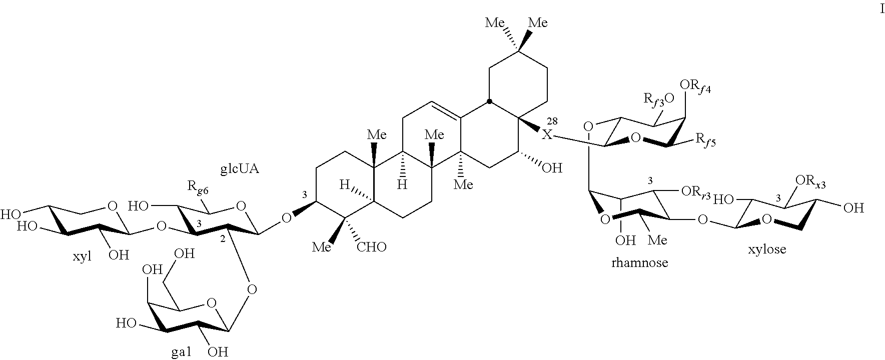

X is —C(O)O—, —C(O)NH—, —(CH₂)ₐO—, —CO₂(CH₂)ₐO—, or —C(O)NH(CH₂)ₐO—, wherein a is an integer from 1 to 5;

R_{g6} is H, Me, alkyl, —COR₁, or —CH₂OR′;

Rₓ₃, Rᵣ₃, and R_{f3} are each independently H or a monosaccharide;

R_{f4} is H, acetyl, or a monosaccharide;

R_{f5} is H, Me, alkyl, monosaccharide, —COR₁, OR′, or —CH₂OR′; where
R₁ is OH, —NR₂(CH₂)ₘR₃, —NR₂[(CH₂)ₙ—O—(CH₂)ₚ]ₓR₃, —NR₂[(CH₂)ₙ—NH—(CH₂)ₚ]ₓR₃, —NR₂[(CH₂)ₙ—S—(CH₂)ₚ]ₓR₃, —NR₂[(CH₂)ₙ—C(O)—NH—(CH₂)ₚ]ₓR₃, or —NR₂[(CH₂)ₙ—NH—C(O)—(CH₂)ₚ]ₓR₃
R′ is H, —(CH₂)ₘR₃, —[(CH₂)ₙ—O—(CH₂)ₚ]ₓR₃, —[(CH₂)ₙ—NH—(CH₂)ₚ]ₓR₃, —[(CH₂)ₙ—S—(CH₂)ₚ]ₓR₃, —[(CH₂)ₙ—C(O)—NH—(CH₂)ₚ]ₓR₃, or —[(CH₂)ₙ—NH—C(O)—(CH₂)ₚ]ₓR₃; and
R₂ is H, alkyl, (CH₂)ₘR₃, —[(CH₂)ₙ—O—(CH₂)ₚ]ₓR₃, —[(CH₂)ₙ—NH—(CH₂)ₚ]ₓR₃, —[(CH₂)ₙ—S—(CH₂)ₚ]ₓR₃, —[(CH₂)ₙ—C(O)—NH—(CH₂)ₚ]ₓR₃, or —[(CH₂)ₙ—NH—C(O)—(CH₂)ₚ]ₓR₃;
R₃ is CH₃, Ph, COOH, CHO, CONH₂, OH, SH, NH₂, a monosaccharide, a disaccharide, a trisaccharide, or a tetrasaccharide; and
m is an integer from 1 to 14, n and p are each independently integers from 0 to 12, and x is an integer from 1 to 12;

### Example 3

A QS-21 derivative having the formula (II):

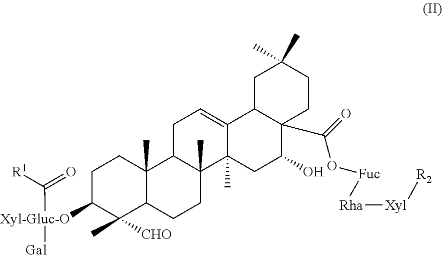

where:

R₁ can be OBn, —NH(CH₂)ₘCH₃, —NH(CH₂)ₙ—O—(CH₂)ₚ, CO₂H—(CH₂)_{q}—NHCO—(CH₂)ᵣ—, CO₂H—(CH₂)ₛ, trisaccharide-(CH₂)ₜ, or CHO—(CH₂)ᵤ, wherein m=can be an integer from 1 to 14; n, p, q, r, s, t and u are each independently integers from 1 to 12,

where, when R₁ is OBn, the xylose and galactose are optionally blocked with triethylsilyl (TES), and where the trisaccharide can be fucose-rhamnose-xylose-R₂, where R₂ can be H, xylose or apiose, and where the fucose-rhamnose-xylose-R₂ can be optionally blocked with acetyl groups (Ac).

### Example 4

An QS-21 derivative having the formula (III):

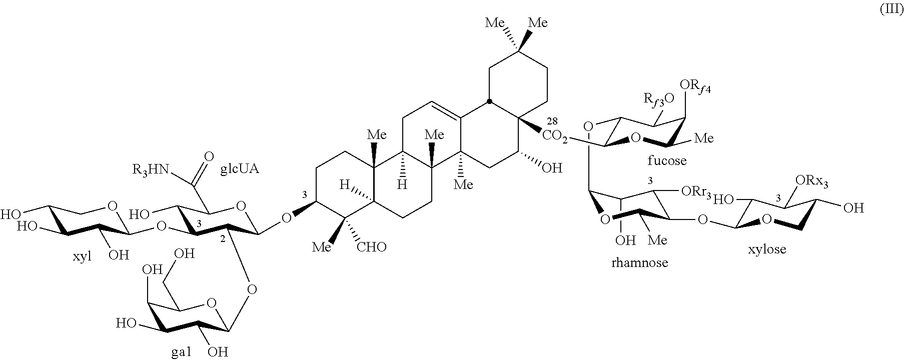

wherein

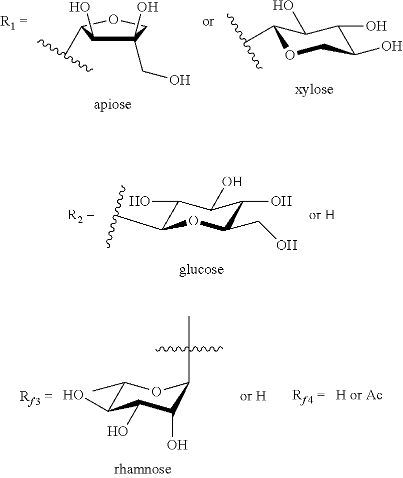

### and R₃ is any of:

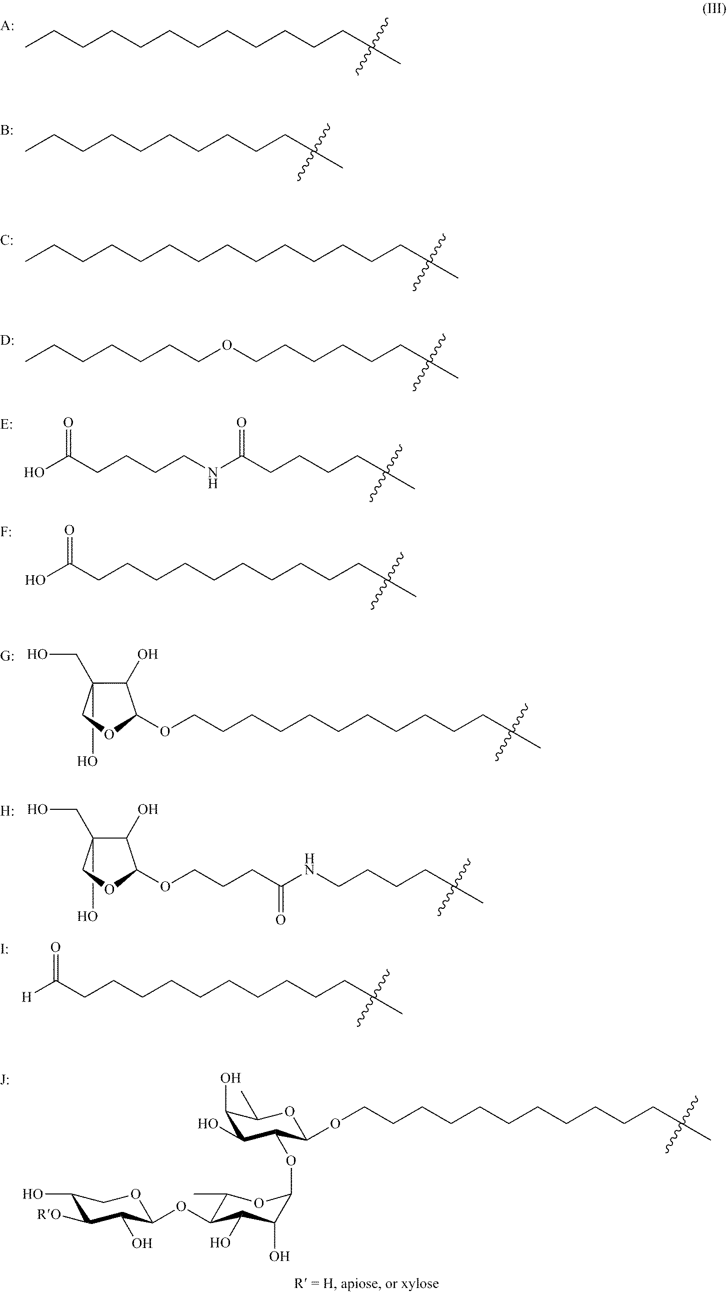

What is claimed:

1. A QS-21 derivative having the formula (I): 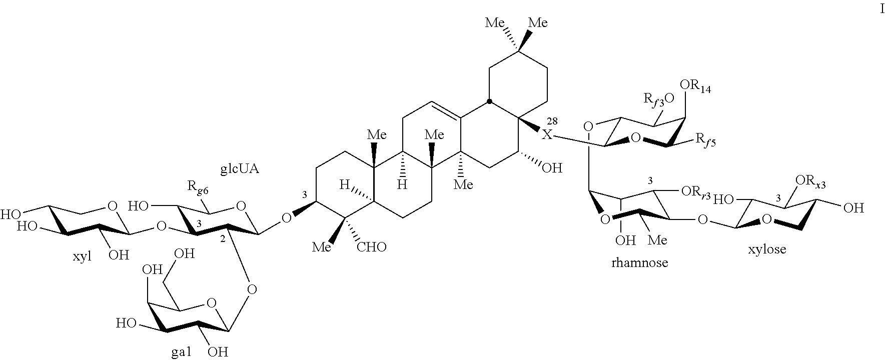
X is —C(O)O—, —C(O)NH—, —(CH₂)ₐO—, —CO₂(CH₂)ₐO—, or —C(O)NH(CH₂)ₐO—, wherein a is an integer from 1 to 5;
R_{g6} is H, Me, alkyl, —COR₁, or —CH₂OR′;
Rₓ₃, Rᵣ₃, and R_{f3} are each independently H or a monosaccharide;
R_{f4} is H, acetyl, or a monosaccharide;
R_{f5} is H, Me, alkyl, monosaccharide, —COR₁, OR′, or —CH₂OR′; where
R₁ is OH, —NR₂(CH₂)ₘR₃, —NR₂[(CH₂)ₙ—O—(CH₂)ₚ]ₓR₃, —NR₂[(CH₂)ₙ—NH—(CH₂)ₚ]ₓR₃, —NR₂[(CH₂)ₙ—S—(CH₂)ₚ]ₓR₃, —NR₂[(CH₂)ₙ—C(O)—NH—(CH₂)ₚ]ₓR₃, or —NR₂[(CH₂)ₙ—NH—C(O)—(CH₂)ₚ]ₓR₃
R′ is H, —(CH₂)ₘR₃, —[(CH₂)ₙ—O—(CH₂)ₚ]ₓR₃, —[(CH₂)ₙ—NH—(CH₂)ₚ]ₓR₃, —[(CH₂)ₙ—S—(CH₂)ₚ]ₓR₃, —[(CH₂)ₙ—C(O)—NH—(CH₂)ₚ]ₓR₃, or —[(CH₂)ₙ—NH—C(O)—(CH₂)ₚ]ₓR₃; and
R₂ is H, alkyl, (CH₂)ₘR₃, —[(CH₂)ₙ—O—(CH₂)ₚ]ₓR₃, —[(CH₂)ₙ—NH—(CH₂)₂]ₓR₃, —[(CH₂)ₙ—S—(CH₂)ₚ]ₓR₃, —[(CH₂)ₙ—C(O)—NH—(CH₂)ₚ]ₓR₃, or —[(CH₂)ₙ—NH—C(O)—(CH₂)ₚ]ₓR₃;
R₃ is CH₃, Ph, COOH, CHO, CONH₂, OH, SH, NH₂, a monosaccharide, a disaccharide, a trisaccharide, or a tetrasaccharide; and
m is an integer from 1 to 14, n and p are each independently integers from 0 to 12, and x is an integer from 1 to 12;

2. The QS-21 derivative according to claim 1, wherein Rₓ₃ is H, apiose or xylose.

3. The QS-21 derivative according to claim 1, wherein R_{f3} is H or glucose.

4. The QS-21 derivative according to claim 1 having the formula (II): 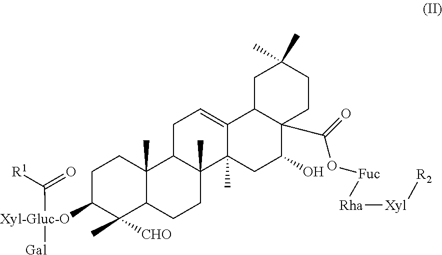
where:
R₁ can be OBn, —NH(CH₂)ₘCH₃, —NH(CH₂)ₙ—O—(CH₂)ₚ, CO₂H—(CH₂)_{q}—NHCO—(CH₂)ᵣ—, CO₂H—(CH₂)ₛ, trisaccharide-(CH₂)ₜ, or CHO—(CH₂), wherein m=can be an integer from 1 to 14; n, p, q, r, s, t and u are each independently integers from 1 to 12,
where, when R₁ is OBn, the xylose and galactose are optionally blocked with triethylsilyl (TES), and
where the trisaccharide can be fucose-rhamnose-xylose-R₂, where R₂ can be H, xylose or apiose, and where the fucose-rhamnose-xylose-R₂ can be optionally blocked with acetyl groups (Ac).

5. The QS-21 derivative according to claim 1, wherein when R_{g6} is —COR₁, and R₁ is selected from:

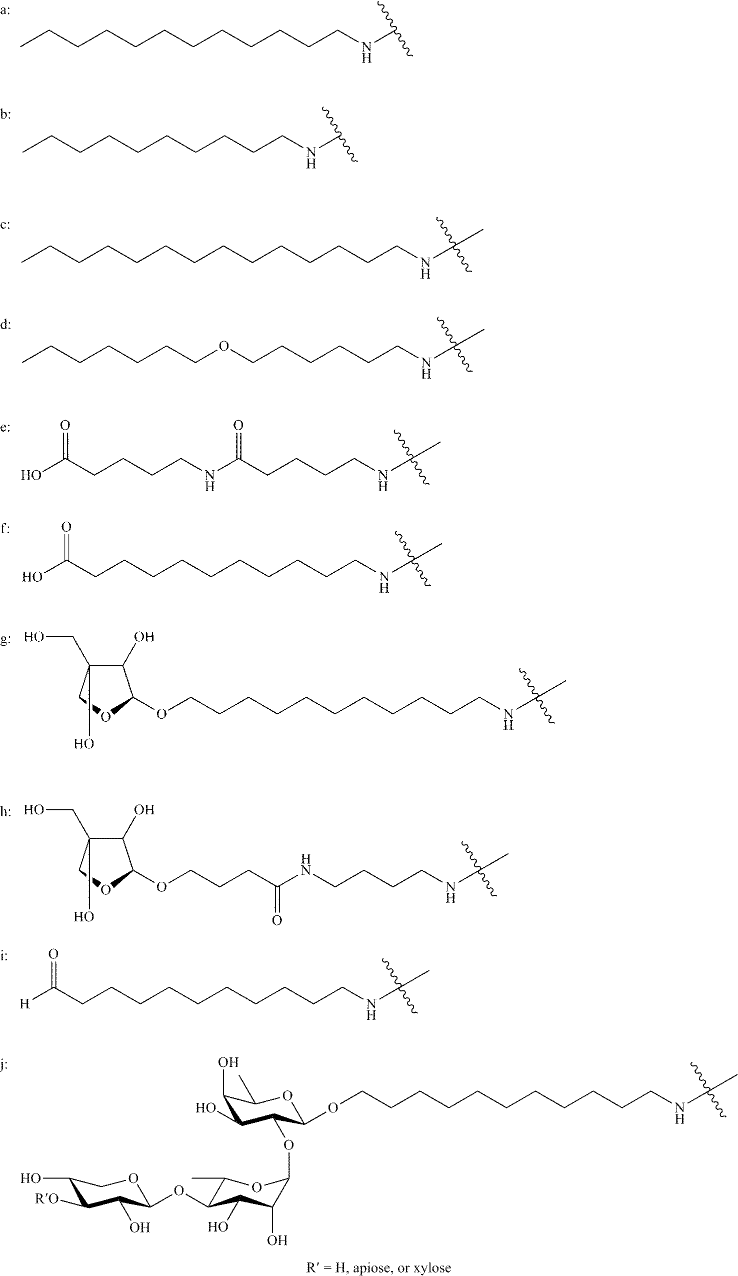

6. A QS-21 derivative having the formula (III): 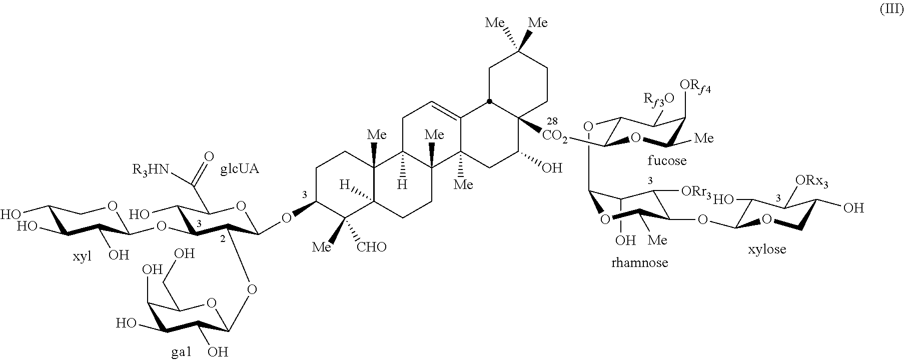
wherein 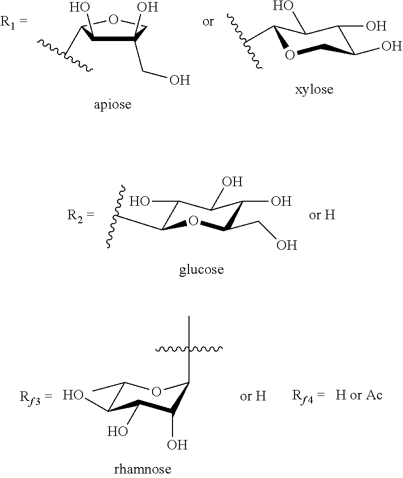
and R₃ is any of:

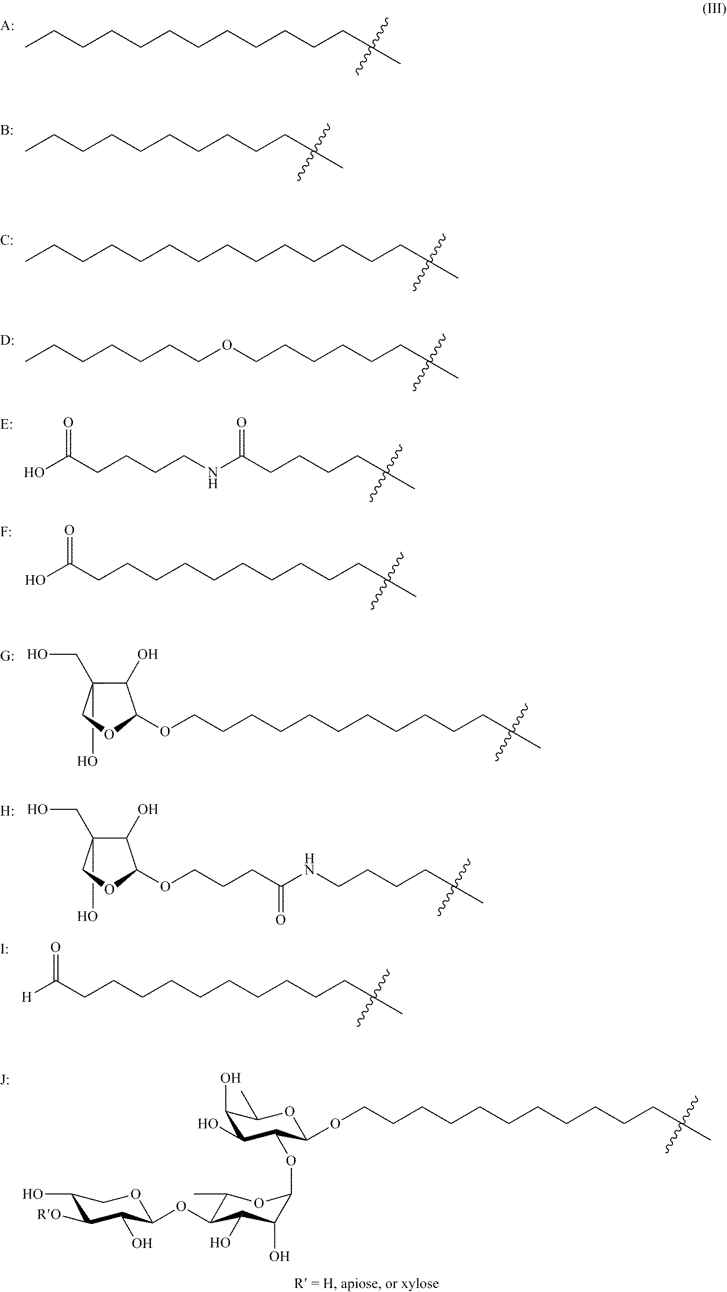

7. A pharmaceutically acceptable composition comprising at least one of the compounds according to claim 1.

8. The pharmaceutically composition according to claim 7, wherein the composition is formulated as a vaccine.
